(19) United States
(12) Patent Application Publication
(10) Pub. No.: US 2017/0100408 A1
(43) Pub. Date: Apr. 13, 2017

(54) IVABRADINE ADSORBATES

(71) Applicant: LABORATORIO CHIMICO INTERNAZIONALE S.p.A., Milano (IT)

(72) Inventors: Giorgio BERTOLINI, Segrate (MI) (IT); Cinzia BIAGGI, Segrate (MI) (IT); Ilaria FERRANDO, Mendrisio (CH) (CH)

(21) Appl. No.: 15/129,207

(22) PCT Filed: Mar. 24, 2015
(86) PCT No.: PCT/IB2015/000357

§ 371 Date: Sep. 26, 2016

(30) Foreign Application Priority Data
Mar. 27, 2014 (IT) MI2014A000524

(51) Int. Cl. A61K 31/55; A61K 9/16

(52) U.S. Cl. CPC A61K 31/55; A61K 9/1611

(57) ABSTRACT

The subject-matter of the present invention is a novel, non-salified ivabradine solid form, in particular an ivabradine form adsorbed on an inert carrier. The subject-matter of the invention is also a process for preparing said solid form, its use in therapy and pharmaceutical compositions comprising it.

### SUMMARY OF THE INVENTION

The subject-matter of the present invention is a novel, non-salified ivabradine solid form, in particular an ivabradine form adsorbed on an inert carrier. The subject-matter of the invention is also a process for preparing said solid form, its use in therapy and pharmaceutical compositions comprising it.

### TECHNICAL FIELD

Ivabradine or 3-[3-({[(7S)-3,4-dimethoxybicyclo [4.2.0]octa-1,3,5-trien-7-yl]methyl}(methyl)amino) propyl]-7,8-dimethoxy-1,3,4,5-tetrahydro-2H-3-benzazepin-2-one, having the following formula

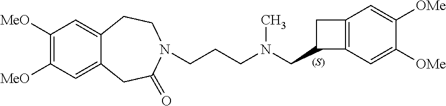

is used in cardiology against heart failure, hypertension, angina and post-infarction treatment and has been described for the first time in Patent EP 0 534 859 in the name of Adir/Servier.

Ivabradine free base is a yellowish, very viscous oil that is hardly treatable and difficult to formulate. For this reason ivabradine base has not been considered convenient for preparing pharmaceutical compositions to be used in therapy and it has been preferred to formulate pharmaceutical compositions containing ivabradine in salified form, in particular as hydrochloride salt, which is a solid and therefore being more suitable to the industrial processing.

However, considering the low dose of active ingredient present in the ivabradine formulates (for example tablets comprising only 5 mg of ivabradine), the dispersion of such a small amount of solid active ingredient in the solid matrix of excipients, with the purpose of obtaining a highly homogeneous dispersion, is not an easy, industrial procedure.

Thus there is the need for a solid form of ivabradine allowing a homogeneous dispersion thereof in the pharmaceutical composition, even when used in low dosage, without this leading to difficulties in industrial processing.

It is known that sometimes is possible to “dilute” the active ingredient in an inert carrier and the obtained composite mixture can then be used in the final dispersion for the formulation.

EP2468258 describes a process for preparing pharmaceutical compositions of compounds poorly soluble in water, which comprises the suspension of the compounds in a solvent wherein said compounds are insoluble, the treatment of the so-obtained suspension with high input of energy, followed by a granulation treatment. Said treatment, applicable according to the document to an infinite number of active ingredients, proves to be very laborious and needs particular and sophisticated equipments.

However, also in this case, the mixing operation of the active ingredient in the carrier must meet important requirements in order to ensure the subsequent effective use of the pharmaceutical composition. In particular, at least two requirements must be ensured:
1. the characteristic of the active ingredient and the process used for the dispersion on the carrier must lead to a dispersion as homogeneous as possible;
2. the ratio carrier to active ingredient must be the lowest possible in order to reduce the impact on the final formulation to the minimum.

The skilled in the art knows that it is not always possible to satisfy both these conditions and obtain a composition suitable to industrial processing and effective administration.

### OBJECTS OF THE INVENTION

It is an object of the invention to provide ivabradine adsorbates on inert supports, presenting in solid form and which can be easily processed and formulated in pharmaceutical compositions.

It is a further object of the invention a process for preparing ivabradine adsorbates, which is industrially simple and does not require particular apparatus.

It is a further object of the invention to provide pharmaceutical compositions, for example tablets, capsules, granulates, etc., comprising the ivabradine adsorbates of the invention.

### DESCRIPTION OF THE INVENTION

According to one of its aspects, the invention relates to ivabradine adsorbates on pharmaceutically acceptable inert solid carriers.

Ivabradine has the following formula (I)

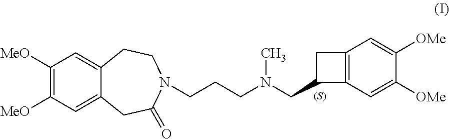

The term “adsorbate” or “adsorbates” herein refers to a solid mixture wherein ivabradine free base is supported on a pharmaceutically acceptable inert solid carrier. By “inert solid carrier” is herein meant any bulking agent (or diluent) used in the pharmaceutical field. Suitable inert, solid carriers include for example (poly)saccharides such as cyclodextrins, starches, maltodextrins, cellulose and derivatives thereof, lactose, mannitol; salts or minerals such as magnesium and aluminum silicates, silica, kaolin, talc, calcium phosphates; some solid polyalcohols such as sorbitol; magnesium salts of fatty acids; zeolites; and pharmaceutically acceptable water-soluble polymers, such as PEG (polyethyleneglycol), PVP (polyvinylpyrrolidone) and the like.

According to the present invention, proteins and derivatives thereof, for example albumin, are not considered inert solid carriers.

According to an advantageous embodiment of the invention, inert solid carriers are selected from silica (silicon dioxide) and magnesium and aluminum silicates.

According to another advantageous embodiment of the invention, inert solid carriers are selected from cellulose and derivatives thereof, such as microcrystalline cellulose, carboxymethylcellulose and hydroxypropylmethylcellulose.

According to another advantageous embodiment of the invention, inert solid carriers are selected from cyclodextrins, for example beta or gamma cyclodextrin, starch, lactose and maltodextrins.

The adsorbates of the invention can comprise ivabradine and one or more pharmaceutically acceptable inert solid carriers. According to a preferred embodiment, ivabradine adsorbates comprise ivabradine and only one inert solid carrier.

With the purpose of obtaining a homogeneous dispersion of ivabradine on the inert solid, the oily characteristic of this active ingredient has been exploited, which allows the preparation of the adsorbates of the invention by dissolving ivabradine base, that is a viscous oil, in a convenient solvent or convenient mixture of solvents, by adding at least one inert solid carrier, cooling and lyophilizing the so-obtained mixture. Alternatively, the mixture comprising ivabradine, solvent or mixture of solvents and the at least one inert solid carrier can be atomized (“spray-dried”), according to methods well known in the art.

Alternatively, the mixture comprising ivabradine, solvent or mixture of solvents and at least one inert solid carrier, can be subjected to a simple solvent evaporation, according to methods also well known in the art.

Suitable solvents are for example alcohols, advantageously C1-C4 alcohols, such as methanol, ethanol, propanols and butanols, for example tert-butanol; ketones such as acetone, methylethylketone, methylisobutylketone, toluene, esters such as ethyl acetate, ethers such as tetrahydrofuran, methyl-tert-butyl ether, or chlorinated solvents such as dichloromethane or mixtures thereof or, in alternative, mixtures of these water-soluble solvents.

Preferred solvents according to the invention are alcohols, advantageously propanols or butanols.

According to another of its aspects, subject-matter of the invention is a process for preparing ivabradine adsorbates comprising:
a. dissolving ivabradine base in a solvent or mixture of solvents;
b. adding one or more inert solid carriers to the solution obtained in step (a);
c. subjecting the mixture obtained in step (b) to solvent evaporation, or alternatively lyophilization or, alternatively, spray-drying.

Advantageously, step (a) is carried out at room temperature.

Generally, the mixture obtained in step (b) is a fine suspension that can be subjected to distillation of the solvent or mixture of solvents.

Alternatively, the mixture of step (b) can be cooled under stirring at a temperature lower than −5° C., for example lower than −10° C., preferably lower than −15° C. The suspension is maintained at said temperatures for some hours, for example 5-30 hours, advantageously about 20 hours, before being further cooled and subjected to lyophilization or being subjected to spray-drying.

Surprisingly, the adsorbates of the invention, advantageously but not necessarily prepared according to the process described herein, constitute a perfectly stable and workable powder also in very low ratios of ivabradine base to inert solid carriers.

In fact it has been observed that the adsorbates of the invention can be prepared in the presence of low amounts of inert carrier, for example in ratios ivabradine to inert solid carriers of about 1:0.5 to 1:5, advantageously about 1:1.5 to about 1:3, for example around 1:2 or 1:2.5.

Since these adsorbates are constituted by a mixture of ivabradine, a highly viscous oil, and a relatively small part of inert solid carrier, the obtaining of a stable and workable powder, having the same physical characteristics of the inert supports, was not predictable and therefore represents an unexpected and surprising result.

Adsorbates obtainable and/or obtained by the process of the invention, in particular adsorbates having the features of the preferred embodiments of the invention, are a further subject-matter of the invention.

In the adsorbates of the invention, ivabradine is present in amorphous form.

Adsorbates of the invention allow to obtain an ivabradine base powder, perfectly stable and processable, that can be used for preparing solid pharmaceutical compositions, suspensions and suppositories, but also buccal patches or even transdermal patches, preferably in addition to one or more conventional pharmaceutically acceptable excipients.

Pharmaceutical compositions comprising the ivabradine adsorbates of the invention are a further subject-matter of the invention, such as also the use in therapy of the adsorbates and pharmaceutical compositions containing them, in particular in the treatment of heart failure, hypertension, angina and in the post-infarction treatment.

The invention also comprises a method of treatment of heart failure, hypertension, angina and post-infarction condition comprising administering, to a subject in the need thereof, an effective amount of an adsorbate of the invention, advantageously in the form of a pharmaceutical composition of the invention.

The pharmaceutical compositions of the invention are particularly suitable for oral administration.

For oral administration, said compositions can be in the form of tablets, capsules or granulates and are prepared according to conventional methods with pharmaceutically acceptable excipients such as binding agents, bulking agents, lubricants, disintegrants, wetting agents; flavoring agents, etc. Tablets can also be coated by the methods well known in the art.

The compositions of the invention are advantageously in the form of dosage units. Preferably, each dosage unit according to the invention comprises an amount of ivabradine (free base) from 1 to 10 mg, for example 4 to 8 mg, advantageously 5 mg and 7.5 mg, together with conventional excipients and additives well known to the person skilled in the art.

Thanks to the stability of the adsorbates of the invention, in the compositions according to the invention, the ivabradine adsorbate can be formulated in combination with further active ingredients. According to a preferred embodiment, in the compositions according to the invention the ivabradine adsorbate is formulated as only active ingredient.

Thereby the invention allows to use ivabradine directly as an active ingredient, the ivabradine being, as mentioned, a hardly treatable oil, without the need to convert it in one of its salts, with the further advantage of formulating in a simple and industrially convenient way, pharmaceutical compositions wherein the active ingredient is homogeneously dispersed.

### EXPERIMENTAL SECTION

### Example 1

### Preparation of an Ivabradine and Silica Adsorbate by Lyophilization

Ivabradine free base (0.76 g) is dissolved in 100 ml tert-butanol at room temperature. 1.76 g of silica (Aerosil® 200 Pharma) is added, the so-obtained colloidal suspension is cooled to −15° C. and is kept under stirring for 30 minutes. It is further cooled to −18° C. for a period of 18 hours. The cooled mixture is dried at −53° C. at 0.168 mbar pressure for 24 hours, thus providing a white powder.

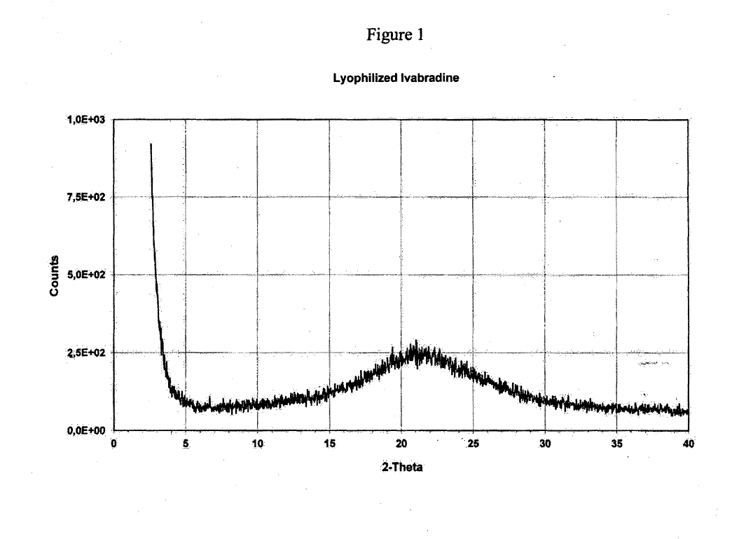

FIG. 1 shows the X-ray spectrum of the ivabradine obtained in Example 1. Adsorbed ivabradine base has not a crystalline form.

1. An ivabradine adsorbate on at least one pharmaceutically acceptable inert solid carrier, wherein the ratio of ivabradine to said at least one solid carrier is 1:0.1 to 1:10 by weight.

2. The adsorbate according to claim 1, wherein said ratio is 1:0.5 to 1:5 by weight.

3. The adsorbate according to claim 2, wherein said ratio is about 1:2 or 1:2.5 by weight.

4. The adsorbate according to claim 1, wherein said at least one carrier is selected from the group consisting of (poly)saccharides, magnesium and aluminum silicates, silica, kaolin, talc, calcium phosphates, solid polyalcohols and magnesium salts of fatty acids; zeolites; and water-soluble polymers such as PEG and PVP and the like.

5. The adsorbate according to claim 4, wherein said at least one carrier is selected from the group consisting of cyclodextrins, starches, maltodextrins, cellulose and derivatives thereof, lactose and mannitol.

6. The adsorbate according to claim 5, wherein said at least one carrier is silicon dioxide (silica).

7. The adsorbate according to claim 1, wherein ivabradine is in an amorphous form.

8. A pharmaceutical composition comprising at least one ivabradine adsorbate according to claim 1.

9. The adsorbate according to claim 1, for its use in therapy.

10. The adsorbate according to claim 1, for its use in the treatment of heart failure, hypertension, angina and post-infarction treatment.

11. A process for preparing ivabradine adsorbates comprising:
a. dissolving ivabradine base in a solvent or mixture of solvents;
b. adding one or more inert solid carriers to the solution obtained in step (a);
c. subjecting the mixture obtained in step (b) to solvent evaporation, or alternatively, lyophilization or, alternatively, spray-drying.

12. The process according to claim 11, wherein said solvent or mixture of solvents is selected from the group consisting of alcohols, ketones, toluene, esters, ethers and chlorinated solvents.

13. The process according to claim 11, wherein said solvent is selected from C1-C4 alcohols.

14. The process according to claim 11, wherein the ratio of ivabradine to solid carrier is 1:0.5 to 1:5 by weight.

15. The process according to claim 11, wherein said one or more carriers are selected from the group consisting of (poly)saccharides, magnesium and aluminum silicates, silica, kaolin, talc, calcium phosphates, solid polyalcohols and magnesium salts of fatty acids; zeolites; and water-soluble polymers such as PEG and PVP.

16. The ivabradine adsorbate obtainable by the process according to claim 11.
